(19) United States
(12) Patent Application Publication
(10) Pub. No.: US 2007/0213584 A1
(43) Pub. Date: Sep. 13, 2007

(54) Percutaneous access and visualization of the spine

(76) Inventors: Daniel H. Kim, Los Altos, CA (US); Singfatt Chin, Pleasanton, CA (US)

Correspondence Address: DANIEL H. KIM, 411 LOS NINOS WAY, LOS ALTOS, CA, 94022, US

(21) Appl. No.: 11/373,848

(22) Filed: Mar. 10, 2006

(51) Int. Cl. A61B 1/00; A61B 17/00

(52) U.S. Cl. 600/104; 600/106; 600/114; 606/1

(57) ABSTRACT

Devices, systems and methods are provided for the percutaneous access and visualization of the spine for the purposes of diagnosing and/or treating a target area of the spine or the surrounding tissue.

### FIELD OF THE INVENTION

The present invention relates to percutaneously accessing and visualizing portions of the spine for the purposes of diagnosing and/or treating a target area of the spine or the surrounding tissue.

### BACKGROUND OF THE INVENTION

The spinal column is formed from a number of bony vertebral bodies 20 separated by intervertebral discs 10 which primarily serve as a mechanical cushion between the vertebral bones, permitting controlled motions (flexion, extension, lateral bending and axial rotation) within vertebral segments. FIG. 1A is a posterior lateral view of two vertebral bodies 20 separated by an intervertebral disc 10. The intervertebral disc 10 is a cushion-like pad with top and bottom endplates 12 adjoining the bone surfaces of each adjacent vertebral body 20. From this posterior vantage point, access to the disc 10 is made difficult by the placement of the disc 10 relative to the vertebral structures such as, the spinous process 60, inferior facet joint 64, superior facet joint 66 and pedicle 67.

FIG. 1B is a coronal view taken through a healthy disc 10 and the surrounding structures. Each endplate 12 (see FIG. 1A) is composed of thin cartilage overlying a thin layer of hard, cortical bone which attaches to the spongy, richly vascular, cancellous bone of the vertebral body 20. The disc 10 includes a nucleus pulposus 30 (“nucleus”), a gel-like substance which acts as a cushion for compressive stress. Surrounding the nucleus 30 is the annulus fibrosis 40 (“annulus”). The annulus 40 includes a number of concentric fibrous layers or sheets of collagen fibers, called lamellae. The annulus 40 limits the expansion of the nucleus 30 when the spine is compressed as well as binds the successive vertebrae 20 together, resists torsion of the spine, and assists the nucleus 30 in absorbing compressive forces. The annulus fibrosis 40 is adjacent annular nerve fibers 80 spinal nerve roots 82, the epidural space 65, the dura 70, the pia or spinal canal 72 and the epidural venous plexus 81.

FIG. 1C shows an exemplary injury 50 to an intervertebral disc 10. In this illustration, the injury 50 is a herniated or prolapsed disc 52. This condition may be the result of a severe or sudden trauma to the spine or nontraumatic pathology, such as degenerative spine disease, may cause a bulge or rupture in one or more intervertebral discs. Through degeneration or injury, the nucleus may become dehydrated becoming less fluid and glutinous. The nucleus may bulge outward causing a reduction in mechanical stiffness of the spinal motion segment which may result in instability.

The annulus 40 is thinnest posteriorly in the general direction of the spinous process 60, so the nucleus 30 usually herniates in that direction. The injury usually proceeds posterolaterally instead of directly posteriorly because the posterior longitudinal ligament strengthens the annulus fibrosis at the posterior sagittal midline of the annulus. The terms “posterior” and “posteriorly” mean the general posterior and posterolateral aspects 43 of the disc as distinguished from the anterior aspects of the disc (i.e., generally in the area of 41).

As illustrated in FIG. 1B, the posterior aspect of the annulus fibrosis 40 is innervated by pain/sensory nerve fibers 80, ventral and/or dorsal nerve roots 82 and other delicate tissues including but not limited to the spinal dura 70. As such, a posterior injury of an intervertebral disc often impinges on one or more of these nerves. The resulting pressure on these nerves often leads to pain, weakness and/or numbness in the lower extremities, upper extremities, or neck region. Additionally, once injured, the healing capacity of the annulus is limited. Usually, healing occurs in the outer layers with the development of a thin fibrous film. However, the annulus never returns to its original strength. In many cases, the annulus never closes becoming highly susceptible to re-herniation or nucleus leakage.

In addition to the traditional bed rest, physical therapy, modifying physical activities, and taking painkillers, there are a growing number of treatments that attempt to repair injured intervertebral discs thereby avoiding surgical removal of injured discs. Many conventional treatment devices and techniques, including open surgical approaches with muscle dissection or percutaneous procedures without visualization, are used to access and penetrate a portion of the disc 10 under fluoroscopic guidance.

One such treatment is disc decompression which involves the removal or shrinking of at least a portion of the nucleus, thereby decompressing and decreasing the pressure on the annulus and adjacent nerves. Techniques and instrumentation have been developed to further lessen the invasiveness of this treatment. Once such technique is automated percutaneous lumbar discectomy (APLD) which employs endoscopy to facilitate visualization to cut nucleus tissue and vacuum away the loosened gelatinous matter. With APLD, however, surgeons cannot observe the nerve root itself (due to the nature of the technique to begin with), and as such, are unable to determine if the nucleus fragments removed are the source of the trouble, nor can they locate and remove any matter that has gone beyond the disc and entered the spinal canal. Another technique to decompressing the disc is microdiscectomy which, as the name implies, involves the use of microscope which magnifies the operative field and provides good lighting. However, a disadvantage of this technique is the inability to recognize adjacent pathology such as a recessed stenosis due to a limited field of vision.

In addition to the removal of disc material, other treatments involve the augmentation of the disc in which devices are implanted in order to treat, delay or prevent disc degeneration. Augmentation refers to both (1) annulus augmentation which includes repair of a herniated disc, support of a damaged annulus, and/or closure of a torn annulus and (2) nucleus augmentation in which additional material is added to the nucleus.

In general, these conventional systems rely on external visualization for the approach to the disc and thus lack any sort of real time, on-board visualization capabilities. Even if a scope is employed, it is limited in its ability to visualize other than what is in its direct course and, even then, without any depth perception to identify the local pathology. While a space may first be created before using the scope, creation of that space, if done percutaneously, is only with external guidance or must be performed blindly.

In addition to the lack of truly effective tools with which to perform the above mentioned procedures and techniques, as observed from the posterior vantage point of FIG. 1A, access to disc 10 is made further difficult by its placement relative to the vertebral structures such as the spinous process 60, inferior facet joint 64, superior facet joint 66 and pedicle 67. Even when the bony structures are able to be navigated, there are other anatomical structures along the access path and/or within the epidural space (such as fats, connective tissue, lymphatics, arteries, veins, blood and spinal nerve roots) which limit the insertion, movement, and viewing capabilities of any access, visualization, diagnostic, or therapeutic device inserted into the epidural space. Further, even if the target space is able to be reached, there is still the risk of damaging nerve roots, the dural sac or other tissue structures along the way.

In sum, many of the conventional procedures for treating the spine (even those considered to be less invasive) do not provide atraumatic direct visualization. As a result, the working space for visualization is limited, there is no ability to visualize, diagnose and treat local pathologies at or adjacent to the target site, and there runs the risk of injury to soft tissue.

Accordingly, a need remains for percutaneous methods and devices which can atraumatically create a working space within tissues, provide percutaneous direct visualization, and enable optimum treatment options. In particular, what is needed are minimally invasive techniques and systems that provide the capability to directly visualize and diagnose or repair a target site within or at the spine while minimizing damage to surrounding anatomical structures and tissues. Moreover, there is still a need for a method and device that allows a physician to effectively enter the epidural space of a patient, clear an area within the space to enhance visualization and use the visualization capability to diagnose and treat the spine injury.

### SUMMARY OF THE INVENTION

The present invention provides devices, systems and methods for accessing and visualizing a target site within the body. They are particularly useful for accessing and visualizing areas of the spine where space is very limited, access is difficult and there involves a high degree of risk of pain or injury to the patient. As such, the devices and systems may be used for any spine related procedure including but not limited to repairing a herniated disc, repairing torn annulus, decompressing the nucleus, implanting annulus or nucleus augmentation devices, implanting electrodes, etc.

An aspect of the present invention is the atraumatic creation of space adjacent a target site, and/or adjacent the distal end of a scope, and/or for the creation of the path or distance between the scope and the target site to provide a perspective view to the user in order to best assess the local pathology and to provide a working space in which to perform a therapeutic or diagnostic task or procedure. In use, the various embodiments of the subject devices and systems employ mechanisms or components to manipulate tissue laterally, distally and/or proximally of the distal end of the device or system. Tissue manipulation as used herein includes various actions upon the tissue including but not limited to moving, pushing, dissecting, compressing, displacing, etc. These manipulations are accomplished by various means in the context of the present invention. In certain embodiments, mechanical members such as frames, struts, wires, hooks, loops, etc. are used, while in others, expandable materials such as inflatable balloons and gel-filled membranes are used.

The novel features, components and devices that enable these inventive aspects are most commonly, but not necessarily, incorporated as part of an access and delivery system or device which may also include known features, components and devices, including but not limited to cannulas, trocars, catheters, guidewires, endoscopes, and working tools for dissecting, removing, cutting, ablating, piercing, suturing, stapling, clipping, irrigating, suctioning, injecting drugs, stem cells and the like, applying energy, sensing, placing electrodes, etc.

Methods are also disclosed for accessing and visualizing a target site within the body, for manipulating tissue and for using the inventive devices and systems.

These and other features, objects and advantages of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying. It is emphasized that, according to common practice, the various features of the drawings are not to-scale. On the contrary, the dimensions of the various features are arbitrarily expanded or reduced for clarity. To facilitate understanding, the same reference numerals have been used (where practical) to designate similar elements that are common to the Figures. Included in the drawings are the following figures:

FIG. 1A is a posterior lateral view of two vertebral bodies; FIG. 1B is a coronal view of a healthy disc and surrounding spinal anatomy; FIG. 1C is a coronal view of a herniated disc;

FIGS. 2A-2D illustrate various views of an embodiment of an access device of the present invention employed with a preformed wire frame type manipulation device of the present invention where the manipulation device is depicted in undeployed and deployed states;

FIGS. 3A and 3B are longitudinal cross-sectional views of an access device employing manipulation device of the present invention including a preformed wire frame/balloon combination where the manipulation device is depicted in undeployed and deployed states;

FIGS. 4A and 4B are longitudinal cross-sectional views of an access device employing a freeform wire type manipulation device of the present invention where the manipulation device is depicted in undeployed and deployed states;

FIGS. 5A-5C illustrate various views of an access device employing a wire manipulation device having preformed spiral or coil configuration where the manipulation device is depicted in undeployed and deployed states;

FIGS. 6A and 6B illustrate undeployed and deployed states, respectively, of another coil-type tissue manipulation device of the present invention integrated with an access device of the present invention;

FIGS. 7A and 7B illustrate undeployed and deployed states, respectively, of yet another loop-type tissue manipulation device of the present invention integrated with an access device of the present invention;

FIGS. 8A and 8B illustrate undeployed and deployed states, respectively, of a balloon-type tissue manipulation device of the present invention method integrated with an access device; FIGS. 8C, 8D and 8E illustrate side and end views of the manipulation device; FIG. 8F illustrates a side view of a slight variation of the manipulation device;

FIGS. 9A-9D illustrate variations of other balloon-type access and manipulation devices of the present invention; FIG. 9E illustrates a manner in which the balloon manipulation devices of the present invention can be employed;

FIGS. 10A-10D illustrate a gel-based manipulation device of the present invention in various acts of deployment and use;

FIGS. 11A-11C illustrate various embodiments of a proximal tissue displacement feature of the present invention; and

FIGS. 12A-12C illustrate various views of an embodiment of a method of performing a therapy in the spinal region using a posterior lateral approach employing the tissue manipulation device of FIGS. 8A-8E and the proximal tissue displacement device of FIG. 11A.

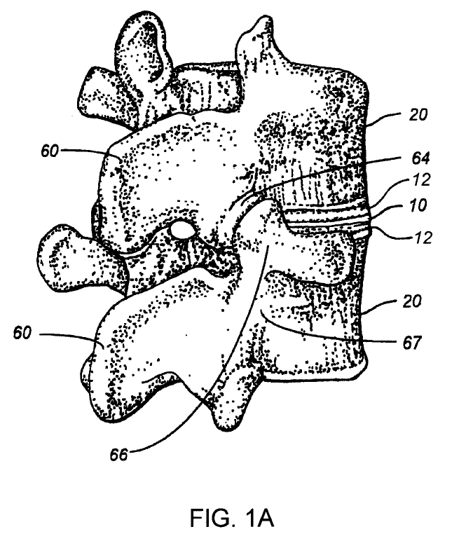

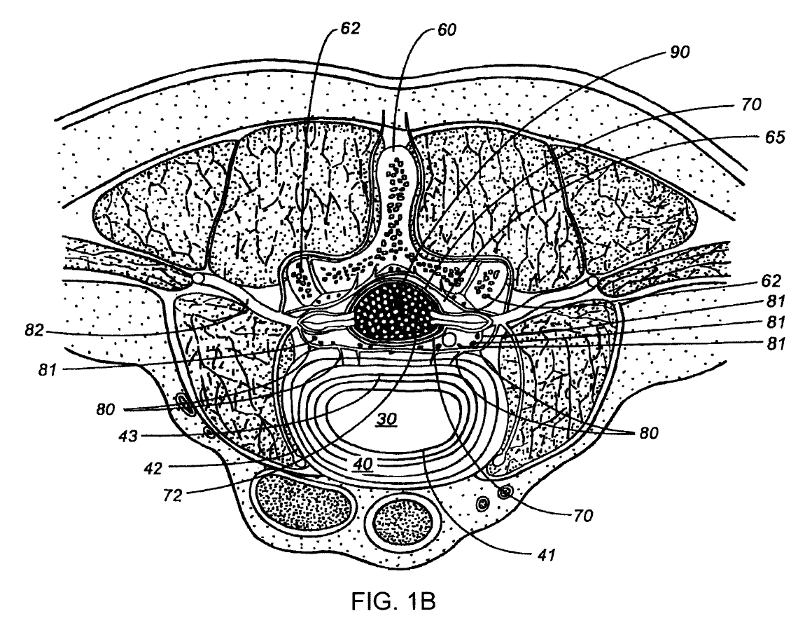

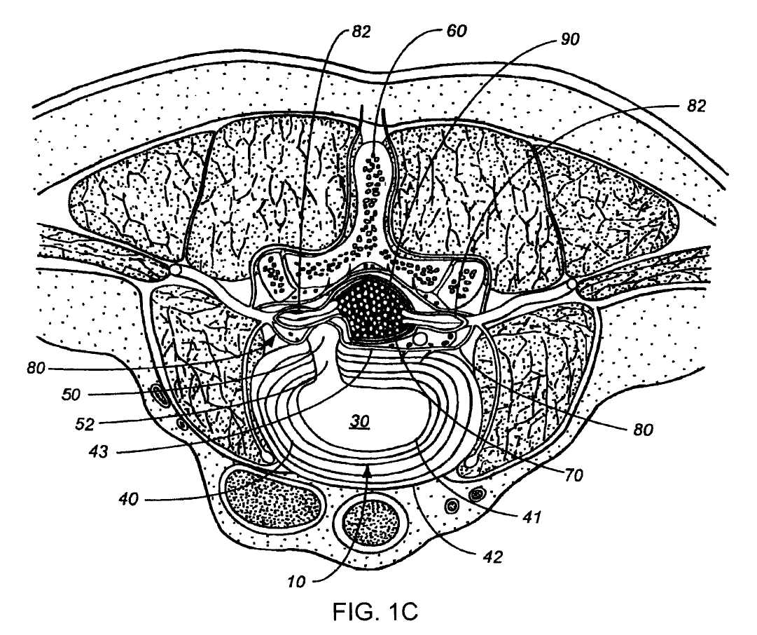

### DETAILED DESCRIPTION OF THE INVENTION

The devices and instruments of the present invention are primarily directed to accessing and visualizing a target site within the body, and are particularly useful for accessing and visualizing areas of the spine where space is very limited, access is difficult and there involves a high degree of risk of pain or injury to the patient. The exemplary application upon which the present invention is described is in the context of the spine and, more particularly, in the context of intervertebral discs. Other exemplary applications to which the subject devices and uses thereof may be employed include but are not limited to cardiac, neurological, vascular, intestinal, reproductive and other applications in which the target surgical site involves delicate organs and soft tissue structures where access is particularly difficult or cumbersome.

The subject devices and instruments may be used in conjunction with or as a component of other known devices and systems. For example, U.S. patent application Ser. No. 11/078,691 filed on Mar. 11, 2005, and U.S. patent application having attorney docket no. SPVW-001CIP filed on Feb. 23, 2006, both entitled “Percutaneous Endoscopic Access Tools for the Spinal Epidural Space and Related Methods of Treatment” and incorporated by reference herein in their entirety, disclose various instruments for accessing, visualizing, diagnosing and/or treating a target site within or at an intervertebral disc or other tissue site within the body which may be employed in whole or in part with the present invention.

An important aspect of the present invention is the atraumatic creation of space adjacent the target site, and/or adjacent the distal end of a scope and/or the path or distance between the scope and the target site to provide a perspective view to the user in order to best assess the local pathology and to provide a working space in which to perform a therapeutic or diagnostic task or procedure. The novel features, components and devices that enable these inventive aspects are most commonly, but not necessarily, incorporated as part of an access and delivery system or device which may also include known features, components and devices, including but not limited to cannulas, trocars, catheters, guidewires, endoscopes, and working tools for cutting, piercing suturing, stapling, clipping, injecting, removing, etc. As such, the terms “access device”, “access system”, “delivery device”, “delivery system” and the like, as used herein, may include one or more known components or devices commonly used in the field of the invention, as well as features, components and devices of the subject invention.

Various exemplary embodiments of the invention are now described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

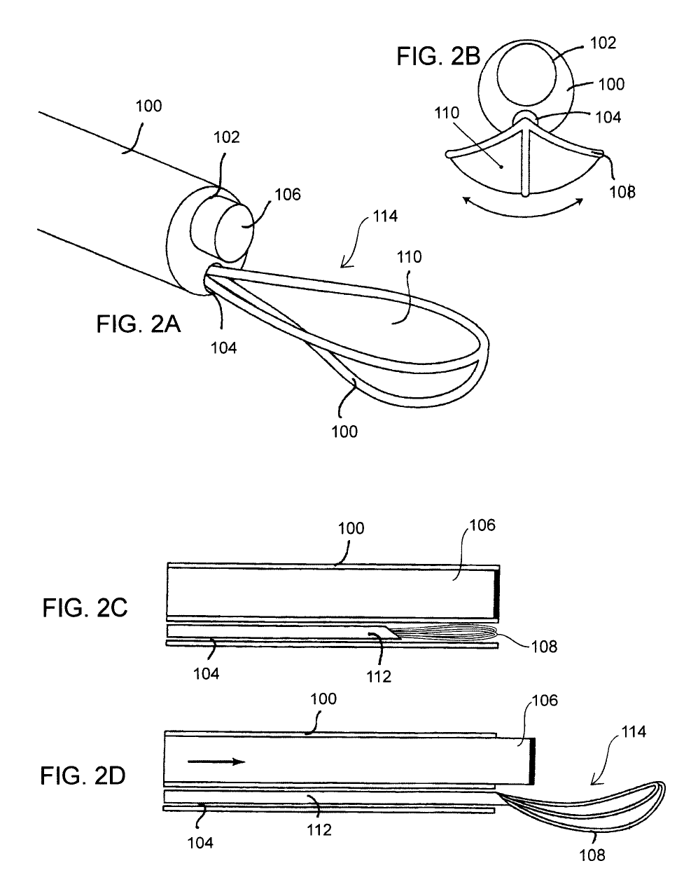

FIGS. 2A-2D illustrate an embodiment of an access device 100 of the present invention. The access device 100 includes a pair of working channels 102, 104 which open at a distal end of device 100, where one of the channels, e.g., channel 102, is a visualization port for the delivery of a scope, imaging and/or illumination components 106 to provide direct visualization capabilities. In an alternative embodiment, rather than a single visualization port housing multiple components, each component may have a dedicated port for illuminating, visualizing, analyzing the surrounding anatomical environment. While visualization port 102 is distally facing or forward looking, in another aspect (not shown), one or more lateral ports may be employed. Tissue differentiating sensors or their functional equivalent may also be provided through the working channels. Additionally, device 100 may be steerable to further enhance its directionality and range of visualization.

A tissue manipulation tool 114 of the present invention having a proximal shaft 112 is provided within and deliverable through the other working channel 104 of device 100. Tool 114 has an open frame structure 108 having struts forming a flower pedal or spoon-like shape where the concave side is inwardly facing, i.e., facing scope 106. The shape (loops, curves, spirals, etc.), surface contours and overall profile of frame 108 are selected to minimize impact when the frame/struts come into contact with anatomical structures, including nerves, muscle and the spinal dura, among others. The wire frame/struts are made of a flexible, conformable material, such as NITINOL or a non-rigid polymer, such that the frame/struts can be compressed to a reduced form for delivery through or stowing within channel 104 (see FIG. 2C) and then allowed to return to an expanded configuration upon exiting or distal advancement from channel 104 (see FIG. 2D). Further, frame 108 may be preconfigured to expand or deploy into any suitable configuration. For example, the convex side of the illustrated frame 108, when in a fully deployed state, extends slightly laterally of the wall of device 100 while minimizing any obstruction within the scope's line of sight. This lateral extension helps to provide “pushback” or resist inward deflection of the frame when abutting anatomical structures and provides maximal working space adjacent scope 106.

When in a fully deployed state, the frame member 108 has a cross-section (best shown in FIG. 2B which provides an end view of the device) defining an arc extending substantially parallel to that of the outer circumference of device body 100 and spanning no more than about 270° and more typically from about 110° to less than about 180°. As such, frame 108 may have a radial dimension (from its central axis to its outer circumference) in the range from about 4 mm to about 10 mm when expanded, and having a maximum linear extension of about 15 mm from distal end of the access device, but may be shorter, wider and longer depending on the application at hand. While a larger arc span is advantageous in displacing a greater mass or volume of tissue than a smaller frame would be able to, a larger frame may require more struts and thereby inhibit visualization by scope 106. Device 114 may be configured such that frame structure 108 is rotatable or swivels within channel 104 (as indicated in FIG. 2B), thereby reducing the size requirements of the frame (and the number of struts required) and allowing a broader visualization range. Additionally or alternatively, shaft 112 and/or tool 114 may be mechanically deflectable or steerable to further enhance visualization and space creation.

Optionally, a webbing material 110 may extend over all or a portion of the open space between the struts to provide additional surface area for displacing, pushing or moving tissue distal to scope 106. Preferably, the web material 110 is transparent so as not to inhibit visualization. Suitable materials for the webbing include but are not limited to polyurethane, silicone and polyester.

Device 100 may have one or more additional working channels for the delivery of any other diagnostic or therapeutic tool or agent which may be used separately or in concert with manipulation tool 114. Examples of other tools and agents that may be delivered through device 100 include but are not limited to sensors, irrigation means, aspiration means, therapy delivery (e.g., RF energy, ablative energy, etc.), drug delivery, implant delivery, cutting means, etc.

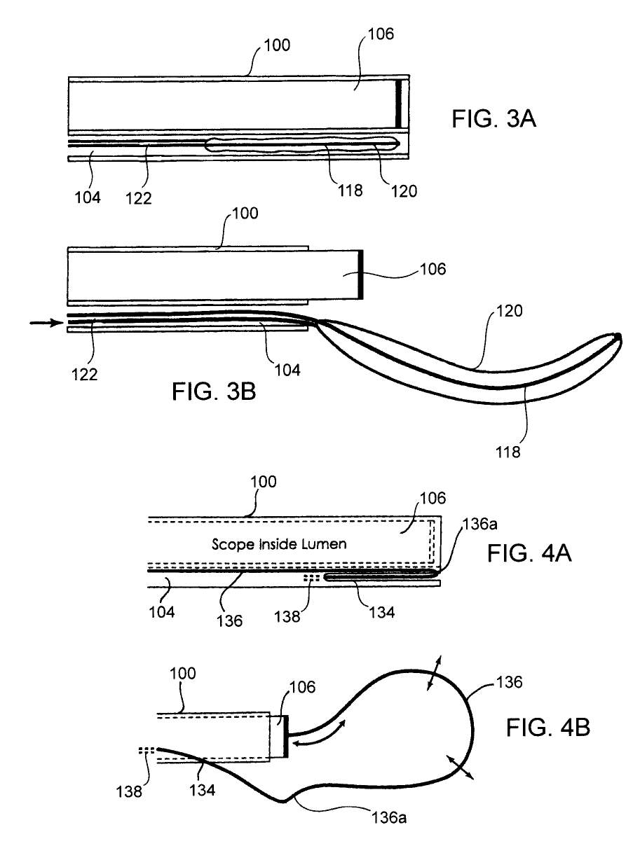

FIGS. 3A and 3B illustrate another embodiment of a tissue manipulation device employed with access device 100 and scope 106. The manipulation device includes an inflatable or expandable balloon 120 affixed to a wire frame 118 and in communication with an inflation/expansion (gas or fluid) lumen 122. As with the manipulation device described with respect to FIGS. 2A-2D, the wire frame 118 is flexible and compressible and may be preconfigured to take on any shape, contouring and profile desired when in an expanded condition. Thus, depending in part on the compliancy of the balloon material, the balloon 120 takes on the general shape of the wire frame 118, as illustrated in FIG. 3B, when unconstrained and inflated. In the stowed position, as illustrated in FIG. 3A, the balloon remains deflated until deployment of the wire frame 118. The balloon material is preferably transparent to allow visualization beyond it.

While the above-described tissue manipulation devices provide a preformed compressible/expandable frame, the frame need not have a preformed shape. For example, the manipulation device 136 of FIGS. 4A and 4B comprises a freeform wire deliverable through working channel 104. One end 134 of wire 136 is anchored at an anchor site 138 to access device 100, such as within lumen 104 or on the exterior surface of the device. From anchored end 134, wire 136 extends distally and, in an undeployed state, as illustrated in FIG. 4A, is bent or folded upon itself at a distance 136a from the anchoring site 138 so as not to extend beyond the distal end of device 100. This configuration provides a flush, low profile front end upon initial percutaneous insertion of the device 100. While the anchor site 138 may be at any location along the length of access device 100 or lumen 104, the closer the anchoring point is to the distal end of the device, the shorter the wire 136 maybe. Minimizing the total length of wire 136 reduces the risk of kinking or crowding.

When undeployed, the remaining wire length extends proximally within channel 104 and exits at a proximal end of device 100 where the free end of the wire (not shown) is available for manipulation. More specifically, the free end is manipulatable to selectively advance and retract wire 136 through lumen 104, as illustrated in FIG. 4B. When deploying the manipulation device, as the length of wire 136 is advanced out of lumen 104, a flexible loop frame is formed, the size of which is readily adjustable by selected advancement/retraction.

The rotational orientation of access device 100 may be adjusted as well to position scope 106 somewhat within the “umbrella” defined by deployed wire 136. Selective manipulation of both the wire and the access device body enables the creation of adequately sized working space into which scope 106 and/or other working tools (not shown) may be advanced to perform the diagnostic or therapeutic task at hand. For example, wire 136 may be incrementally expanded in a distal direction which creates a delivery space for device 100 to move into, the extent of further manipulation of tissue and advancement of device 100 and/or other instrumentation is assessed with information provided by scope 106. The various manipulations, visual assessments and tool advancements are reiterated as necessary to access the intended target site, create a working and visualization space about the target site, and assess the local pathology to determine the specific course of action to be taken, i.e., the type of therapy (e.g., discectomy, annulus augmentation, energy to be applied, etc.) to be performed, the type of diagnostics to be implemented, etc.

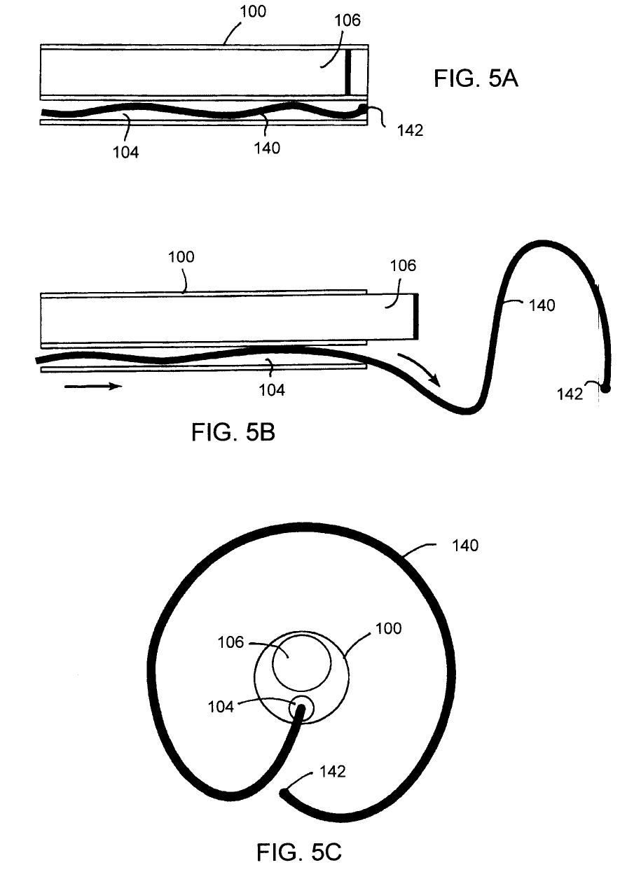

FIGS. 5A-5C illustrate another wire-type manipulation device 140 in use with access device 100. While wire 140 has a preformed shape, neither of its ends is fixed or anchored. Made of a superelastic metal alloy or a flexible polymer like any of the wire type manipulation devices disclosed herein, wire 140 is provided with a preformed spiral or coil shape to which it expands to when deployed, as illustrated in FIGS. 5B and 5C, and is substantially stretched when constrained within port 104, as illustrated in FIG. 5A. The resulting coil has a winding density/spacing to be sufficiently stiff yet flexible to atraumatically create space within tissue. The winding diameter is large enough, i.e., greater than the diameter of scope 106 and typically greater than the diameter of access device 100, so as to allow adequate viewing or and access to areas distal to its distal end 142. The winding diameter may be constant or vary along the wire's length when in an expanded or deployed state. In one variation, as illustrated, the expanded spiral has a diameter which tapers or is reduced from a distal end 142 to a proximal end (not shown).

While the above-described tissue manipulation devices are components which are relatively independent of the access device used to deliver them, in certain invention variations, the manipulation devices are structurally integrated with the access device body. Examples of such an integrated instrument are now described.

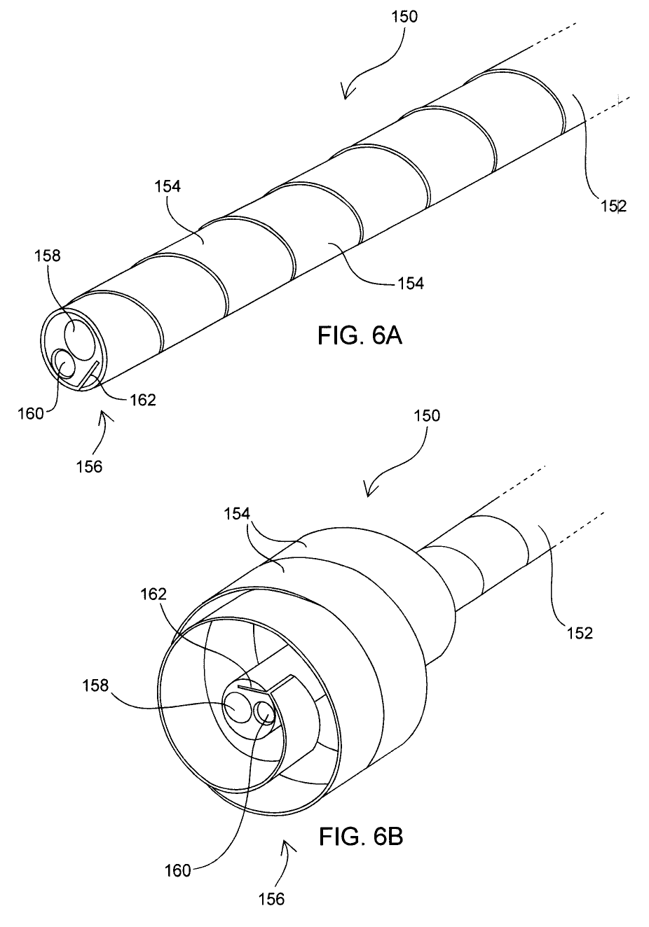

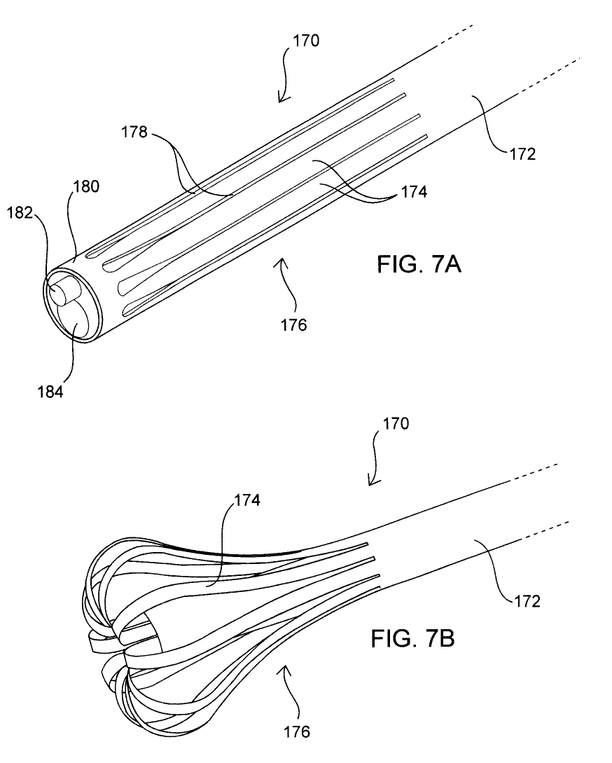

With the embodiments illustrated in FIGS. 6A/6B and 7A/7B, at least a distal portion of the shaft of an access device carries a radially expandable tissue manipulation member. In an undeployed state, as illustrated in FIGS. 6A and 7A, the manipulation member is flush with the outer surface of the access device. In a deployed state, as illustrated in FIGS. 6B and 7B, the manipulation member extends radially from the access device to displace or dissect tissue 360° about the distal end of the access device.

Access device 150 of FIGS. 6A and 6B provides a tissue manipulation member 156 which comprises a wire or ribbon coiled or wrapped around a distal portion of shaft 152. Access device 150 may provide any number of channel lumens 160 for delivering a scope 158 and any other therapeutic or diagnostic tool or agent. Ribbon 156 has multiple windings or bands 154 tightly wound about shaft 152 to provide a flush finish along the shaft's outer surface to facilitate delivery through tissue prior to deployment of member 156. Ribbon 156 may extend (i.e., wind) proximally along the shaft any suitable distance, but typically, only the very distal portion of the shaft need be covered. Radial expansion of the bands 156 is effected by loosening the hold at the proximal end of the ribbon or by the application of heat if made from a temperature sensitive superelastic material. As such, the bands encircle and are substantially orthogonal (with a slight pitch if desired) to the distal portion of shaft 152 The very distal end 162 of ribbon 156 is affixed or anchored to the distal end of shaft 152 so as to maintain control on the extent of radial expansion.

Access device 170 of FIGS. 7A and 7B provides a distally situated tissue manipulation member 176 including a plurality of axially extending bands, struts or stays 174 formed by slots 178 within the tubular material forming manipulation member 176. Struts 174 lie parallel to the longitudinal axis of access device 170. As within any of the subject access devices, device shaft 172 may provide any number of channel lumens 184 for delivering a scope 182 and any other therapeutic or diagnostic tool or agent. Member 170 may extend proximally along the shaft any suitable distance, but typically, only the very distal portion of shaft 172 need be covered. The distal end 180 of member 176 or its respective bands 174 is affixed or anchored to the distal end of shaft 172. Radial expansion of bands 174 is effected by axially moving member 170 and shaft 172 relative to each other. This can be accomplished by moving only shaft 172 in a proximal direction, moving only member 176 in a distal direction or moving both in opposite directions. Alternatively, if made from a temperature-sensitive superelastic material, the bands are expanded by the application of heat. In either case, the stays are caused to expand radially and distally while remaining parallel to the access device 170, as illustrated in FIG. 7B. The fully expanded bands form respective loops with a collective configuration having a donut shape with a central passage through which scope 182 has an unobstructed view. Thus, while moving, pushing or dissecting soft tissue away from the distal end of shaft 172, a distally extending passage is established to provide a working space and perspective visualization by means of scope 182.

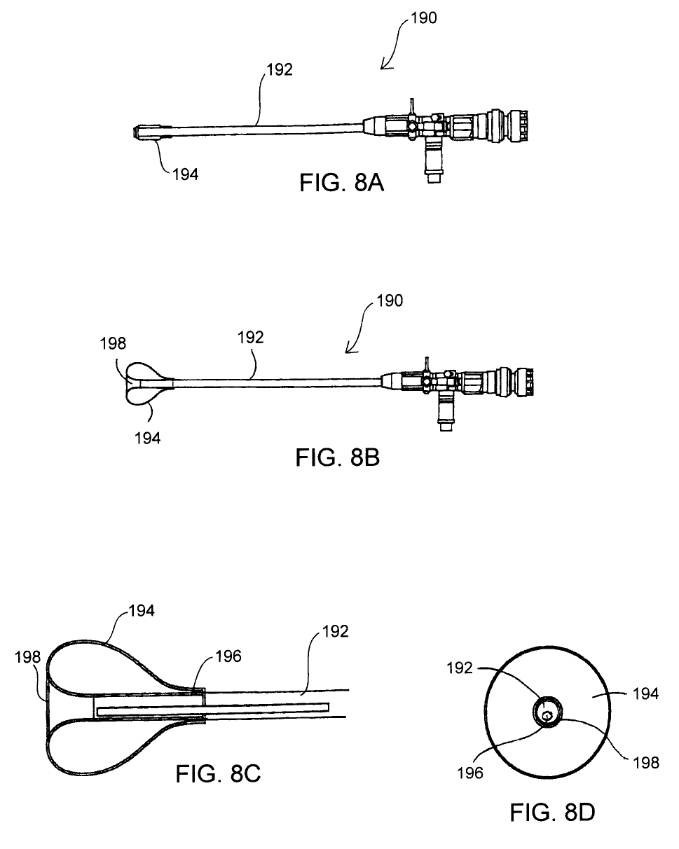

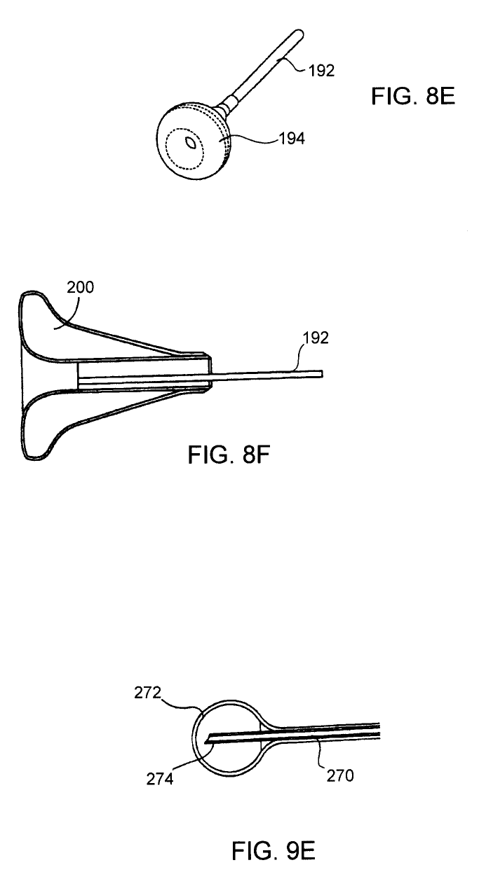

FIGS. 8A-8F illustrate another integrated access system 190 of the present invention employing a balloon-type tissue manipulation device 194. System 190 includes an integrated scope or camera 196 extending through a main shaft 192. Manipulation balloon 194 is in fluid communication with an inflation/expansion means (not shown) integrated within shaft 192. Balloon 194 has a donut configuration which is affixed about the distal end of shaft 192 such that its central hole or opening 198 is aligned with the working channel of shaft 192. As best illustrated in the enlarged side and end views of FIGS. 8C and 8D, the line of sight of scope 196 remains open and unobstructed when balloon 194 is inflated. Depending on the compliancy of the balloon material used, the outer profile of the balloon may be varied. Further, a single balloon may be made multiple portions having variable compliancy. With a material having relatively greater compliance, the inflated balloon has the profile more similar to that of balloon 194 illustrated in FIG. 8C. With a less compliant material, the balloon has a profile more similar to balloon 200 illustrated in FIG. 8F. With either configuration, the balloon moves tissue and clears a working passage/space in a manner similar to that of the mechanically expandable struts of the tissue manipulation member of FIGS. 7A and 7B.

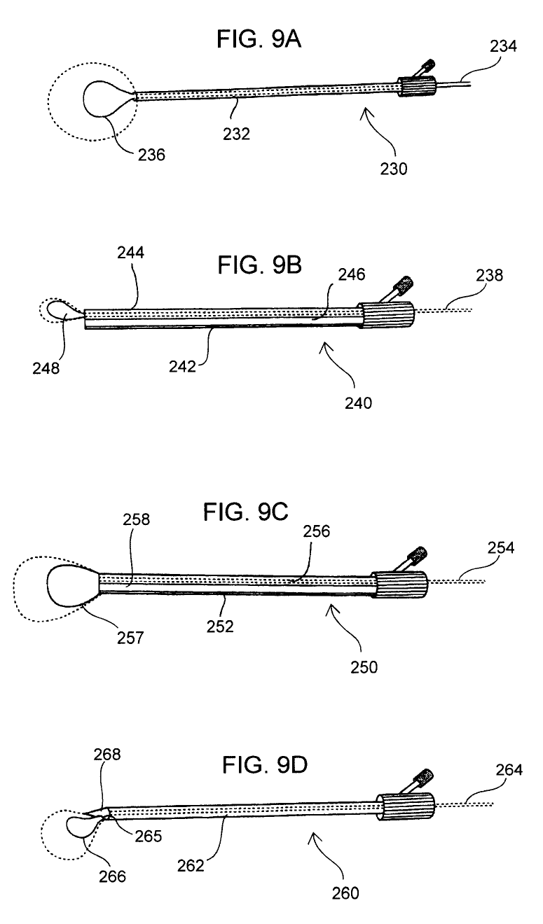

FIGS. 9A-9E illustrate variations of other balloon-type tissue manipulation/access devices. Instrument 230 of FIG. 9A is an endoscope having a single lumen/inflation port for delivery of a scope 234 and selective expansion of a manipulation member 236 comprising a transparent balloon. Balloon 236 is mounted over the distal opening of lumen 232 and, as such, is able to internally receive and encase the distal end of scope 234. The more the balloon is expanded, the further scope 234 can extend distally within tissue without having to further advance shaft 232 into the body. With this configuration, the scope is never exposed to the in vivo elements, unless otherwise desire (as will be explained in greater detail below with respect to FIG. 9E).

Instrument 240 of FIG. 9B includes a dual lumen shaft 242. In addition to a dual purpose scope delivery/balloon inflation lumen 244 for delivery of scope 238 and inflation of transparent balloon 248, shaft 242 includes at least a second working channel 246 for the delivery of other therapeutic and/or diagnostic tools and agents. In use, scope 238 is preferably kept proximally of the expanded balloon such that the delicate dissection is done with the balloon alone. When dissection has been completed and an adequate working/visualization space created, the balloon 248 may be removed if so desired. Notwithstanding, shaft 242 can be axially rotated as necessary to adjust the location of the tissue manipulating member 248 and working channel 242.

Instrument 250 of FIG. 9C has a similar dual lumen shaft 252 construct as that just described; however, the balloon manipulation member 257 extends over the openings of both channels 256, 258. As such, a larger, more centrally positioned working space is created by balloon 257. An even greater difference than the previously described access device is that tools and agents delivered through working channel 252 are not able to directly contact tissue while balloon 257 is in operative use. Accordingly, a feature of this embodiment includes the ability to rupture or break open balloon 257. This would typically be done upon reaching the intended target site after incremental displacement of tissue by balloon 257 and advancement of scope 254. After assessing the local pathology at the target site with confidence of the therapy need to be performed, balloon 257 may be intentionally ruptured to provide direct assess to the target site. Rupturing may be accomplished either by use of scope 254, of a therapeutic instrument delivered through working channel 252 or by over-expansion/inflation of balloon 257. An example of a rupturing means is illustrated in FIG. 9E in the form of a tool 270, which is used as an inflation lumen for balloon 272 and may be used for the delivery of other tools. Tool 274 is provided with a relatively sharp distal tip 274 to easily puncture balloon 272.

While scope 254 may still be employed for visualization subsequent to rupture of the balloon, it may not be needed where the treatment to the target site can be performed “blind.” For example, where the objective is the delivery of a therapeutic agent, the expansion fluid may be the agent itself, where the agent is used to both expand the balloon for creating a working space and then to over-expand the balloon to rupture it whereby the agent is released at the target site.

FIG. 9D illustrates yet another variation of an access device 260 having an integrated balloon-type manipulation member 266. Shaft 262 provides a single lumen for receiving scope 264 and expansion/inflation of balloon 266; however, here, scope 264 is not advancable beyond the distal tip of shaft 262. Instead, a clear tip 268 is provided over the shaft lumen which contains a side port 265 through which balloon 266 is expanded. Tip 268 may be pointed and sharp to function similarly to a trocar in creating a passage/working space for advancement of shaft 262. Tip 268 may also be used to rupture balloon 266 for the delivery of other tools which are required to come into direct contact with the target site.

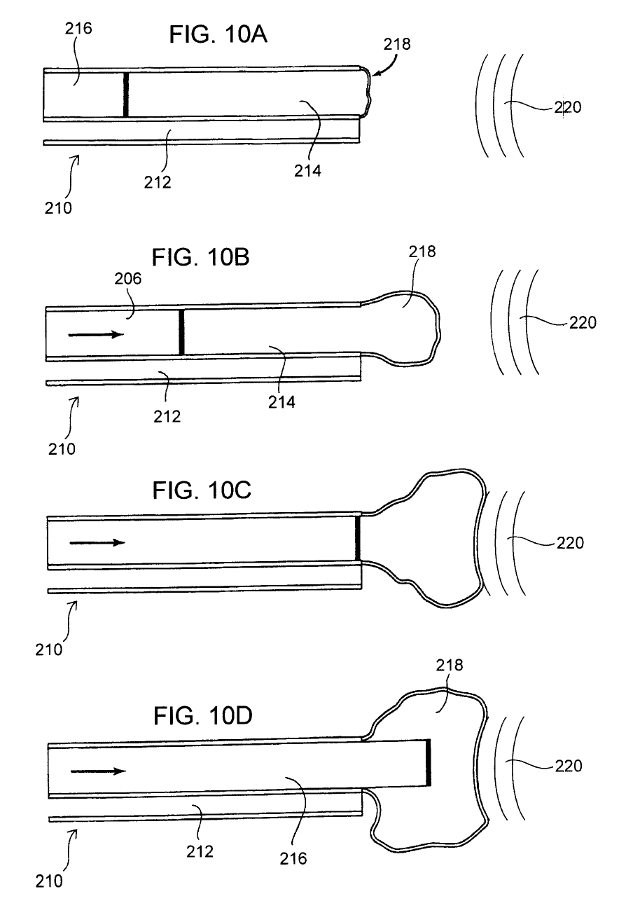

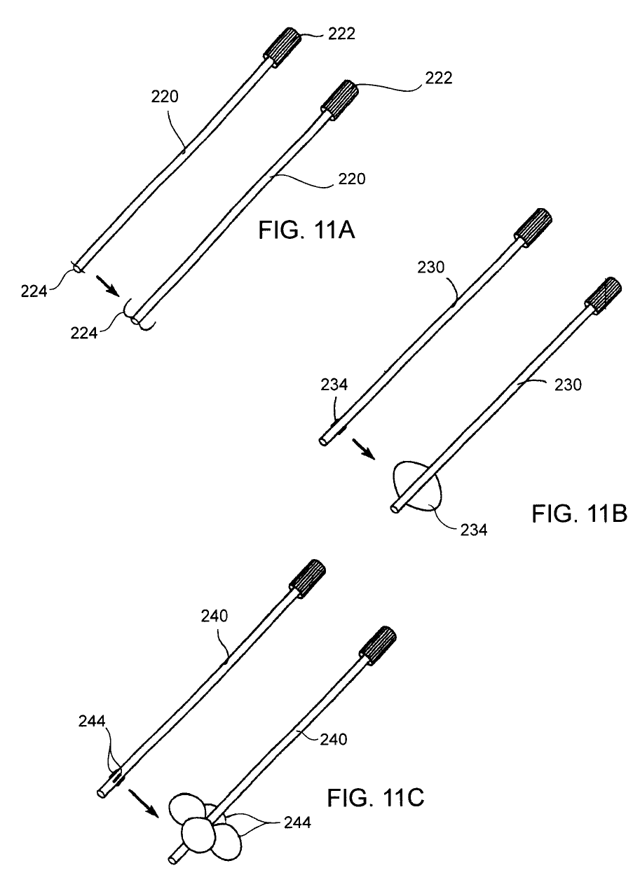

FIGS. 10A-10D illustrate another access device 210 of the present invention which utilizes a clear gelatinous material 214 retained by a transparent, compliant membrane 218 for tissue manipulation. Both the gel and membrane are made of biocompatible materials such as hydrogel and polyurethane, respectively. Gel 214 is initially contained within the distal portion of the lumen used to deliver a scope 216. In other variations, a pusher mechanism (not shown) may be used within the gel-filled lumen to advance the gel material distally; in which case one or more other working channels 212, provided for the delivery of diagnostic and/or therapeutic instruments or agents, may be used for delivery of the scope. With either embodiment, a gasket or other seal (not shown) may be provided within the gel lumen to prevent back flow of the gel from the proximal end of the lumen. With membrane 218 sealed across and covering the distal opening of the lumen, gel 214 is retained within the confines of the lumen, as illustrated in FIG. 10A.

Upon delivery of access device 210, where the distal end of the device is positioned a relatively short distance, from about 2 mm to about 10 mm from a targeted tissue site 220, scope or pusher 216 is distally advanced thereby pushing gel 214 from the lumen. Membrane 218 is sufficiently flexible yet durable to stretch distally to accommodate the extruding gel, as illustrated in FIG. 10B. As scope 216 is advanced, the gel continues to extrude from the device and the membrane continues to stretch to accommodate the extruded volume of gel to the extent that the gel-filled membrane abuts the tissue 220, as illustrated in FIG. 10C. With the resistance of the tissue structure 220 against the membrane 218, the trapped gel 214 expands laterally and displaces fluids and other structures to define an enlarged visualization space into which the distal end of scope 216 can be advanced, as illustrated in FIG. 10D. The formed visualization space provides the user the perspective necessary for a thorough assessment and analysis of the local pathology adjacent target site 220. When direct contact with tissue is necessary by other instrumentation, the gel-filled membrane may itself be pushed or manipulated out of the way by a tool delivered through working channel 212, and as such, continues to provide a clear view for scope 216. When the procedure is complete, proximal retraction of scope 216 creates a negative pressure on the gel and draws it back into the scope lumen. Alternatively, the membrane may otherwise be punctured by use of a working tool whereby the gel is allowed to escape, thereby transforming the visualization space to a working space. With the latter variation, the gel may comprise antibiotic or therapeutic agents to facilitate healing of the target site.

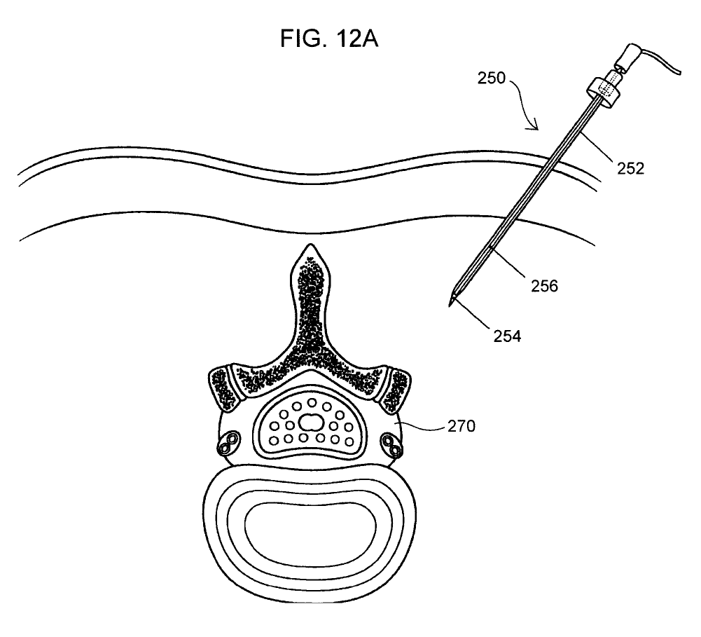

In addition to creating space distally and laterally of the leading or distal tip of an access device, delivery device, scope or other instrument, the present invention also provides for the creation of space proximally of the leading/distal device end. The various tissue manipulation mechanism and components for the proximal space creation can be used independently or collectively with those used for lateral and distal space creation, or otherwise be integrated therewith. FIGS. 11A-11C illustrate examples of such proximal tissue manipulation mechanisms incorporated into an access/delivery tube or cannula which may include a number of channels for the delivery of a scope and therapeutic and diagnostic instruments and agents, or may otherwise be used as an outer sheath through which these components or an inner tube or cannula is deliverable. With any embodiment, the proximal tissue manipulation members may be used solely to displace or dissection tissue and/or may be used to establish traction for the access device while it is in use within the body.

Access device 220 of FIG. 11A employs one, two or a plurality of wire members or hooks 224 which are laterally extendable from the distal end of access device 220. Deployment may be activated, for example, by rotation of a knob 222 positioned at the proximal end of the access device which is attached to pull/push wires or the like housed within the access device. The hooks, when deployed, are driven into adjacent tissue. The hooks may then be used to proximally pull tissue away from the distal end of access device 220 to allow for better visualization with a scope or better access with a working tool.

The proximal tissue manipulation component of access device 230 of FIG. 11B is an inflatable/expandable balloon 234 which is positioned a bit proximally of the distal end of the device and expandable laterally thereof. Balloon 234 is in fluid communication with an inflation/expansion lumen within device 230. In a similar manner, access device 240 of FIG. 11C employs a plurality of balloons 244 to manipulate tissue proximally of the distal end 240. The balloon-type embodiments may be used similarly as the above-described hook-type embodiment in that proximal translation, i.e., pulling, of the access device can create further dissection and/or provide traction.

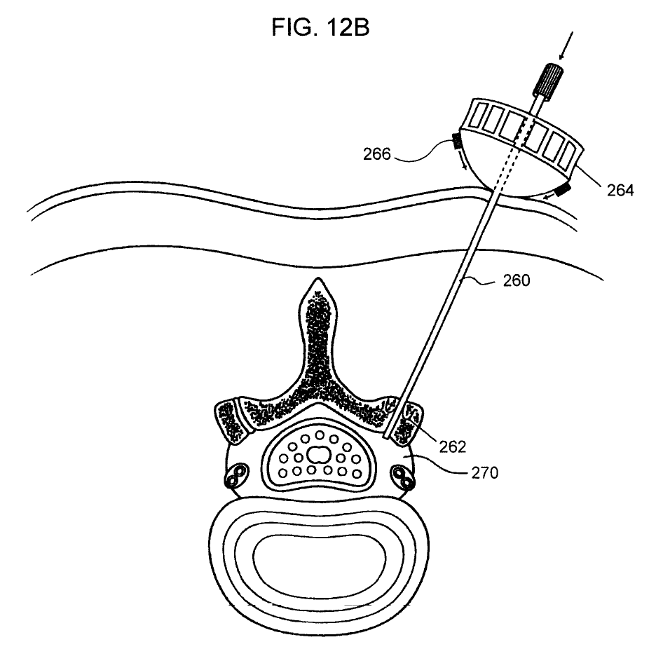

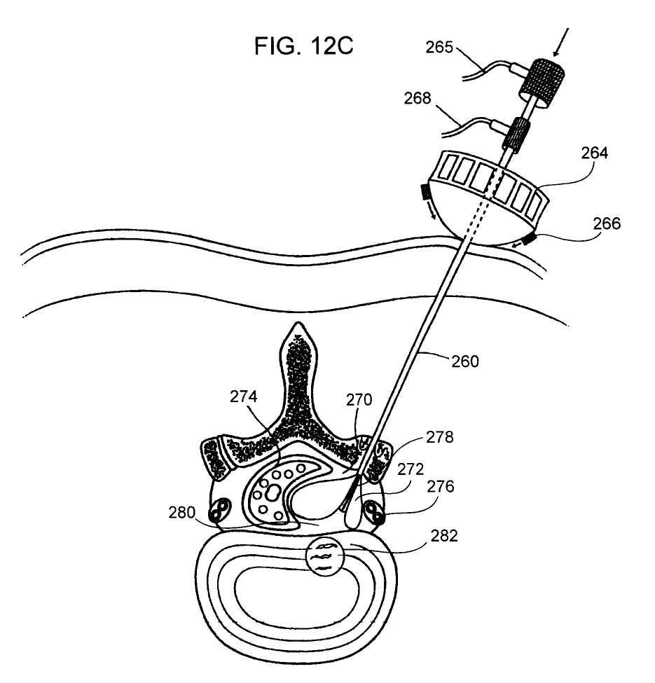

An exemplary method of the present invention is now described with reference to FIGS. 12A-12C and in the context of accessing an intervertebral disc from a posterior or a posterior-lateral approach. An access device 250 including a cannula 252 through which a clear-tipped trocar 254 is delivered and used to create a percutaneous entry through the patient's back as illustrated in FIG. 12A. Fluoroscopy may be used to facilitate this step. A very small diameter scope 256 (having an outer diameter of less than about 1 mm) is delivered through cannula 252. The clear tip 254 of the trocar allows visualization as the access device penetrates through skin, fat and muscle, and as it eventually enters the spinal canal space 270, as illustrated in FIG. 12B, with scope 256 enabling accurate placement therein. At this point in the procedure, the trocar may be removed from the cannula. The scope as well may be removed, however it may be retained within the cannula to facilitate the remainder of the procedure and if not otherwise blocking a working channel for the passage of other instrumentation. Cannula 252, if configured for tissue manipulation, is retained within the back for the duration of the procedure. Alternatively, cannula 252, if a conventional cannula, may simply be used to establish access to within the vicinity of the target site and to deliver a separate space creation device or cannula, such as device 260 depicted in FIG. 12B.

Device 260 (or device 250) includes a scope delivery channel (as well as other working channels) and is equipped with both distal/lateral and proximal space creating mechanisms, although only one of the two may be used. In order to establish traction and/or to create an initial space, the proximal tissue manipulation mechanism 262 (here, in the form of the hook-type device of FIG. 11A) is deployed into tissue just proximal of the distal end of device 260. The hooks 262 are deployed by pushing on actuators 266 positioned about knob 244 proximally mounted to cannula 260. By turning knob 244 tension is placed on the hooks 262 causing them to pull back on the engaged tissue. As such, cannula 260 is stabilized and tissue adjacent the distal end of the cannula is off-loaded a bit so as to facilitate additional tissue manipulation provided by deployment of the distal/lateral tissue manipulation mechanism 272 (here, in the form of balloon-type device of FIGS. 8A-8E), as illustrated in FIG. 12C. Inflated balloon 272 displaces the fatty tissue, dura 274 and nerve roots 276 within the spinal canal space 270, thereby creating a space 280. The distal tissue displacement allows a scope 278 to be advanced distally within visualization space 280 through which to visualize the local pathology. Upon assessment of the area, an optimized treatment course of action may be determined. For example, a torn disc annulus 282, as illustrated in FIG. 12C, is observed. The necessary tools 265, 268 (e.g., blades, suction, irrigation, etc.) may be selected and deployed through the various working channels (not individually shown) to within working space 280. The annulus repair procedure may be visualized by scope 278. Upon completion of the repair, the instrumentation is removed, the tissue manipulation/space creation devices are retrieved, and the access device removed from the patient's back.

In addition to the methods or portions there of described herein, the invention includes methods and/or acts that may be performed using the subject devices or by other means. The methods may all comprise the act of providing a suitable device or system. Such provision may be performed by the end user. In other words, the “providing” (e.g., a delivery system) merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as those generally known or appreciated by those with skill in the art. The same may hold true with respect to method-based aspects of the invention in terms of additional acts as commonly or logically employed.

In addition, though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language. That being said, we claim:

1. A method of percutaneously accessing a target site within the body, the method comprising:
introducing into the body an access device comprising a tissue manipulation member and having direct visualization capability, wherein the tissue manipulation member is deployable from a distal portion of the access device;
deploying the tissue manipulation member thereby displacing tissue adjacent the distal end of the access device to create a space within the tissue between the distal end and the target site; and
directly visualizing the space.

2. The method of claim 1, wherein the tissue manipulation member is deployed in a distal direction.

3. The method of claim 1, wherein the tissue manipulation member is deployed in a lateral direction.

4. The method of claim 1, wherein the tissue displaced by the tissue manipulation member is distal to the distal end of the access device.

5. The method of claim 1, wherein the tissue displaced by the tissue manipulation member is lateral to the distal end of the access device.

6. The method of claim 1, wherein the tissue displaced by the tissue manipulation member is proximal to the distal end of the access device.

7. The method of claim 6, wherein the tissue displaced by the tissue manipulation member is also distal or lateral to the distal portion of the access device.

8. The method of claim 1, wherein the target site is at the spine.

9. The method of claim 8, wherein the target site is an intervertebral disc.

10. The method of claim 1, further comprising delivering an instrument to within the space.

11. The method of claim 1, further comprising deploying a second tissue manipulation member from the access device to stabilize the access device within the body.

12. A method of percutaneously treating a target site within the body, the method comprising:
percutaneously accessing an area of tissue adjacent the target site with an access device having a scope;
expanding a material from the a distal portion of the access device wherein a space is created adjacent the distal portion;
visualizing the space with the scope; and
advancing an instrument to within the space to treat the target site.

13. The method of claim 12, wherein the material defines a balloon.

14. The method of claim 12, wherein the balloon defines a central opening when expanded and the method further comprises advancing the scope through the opening.

15. The method of claim 14, wherein the balloon is expanded substantially laterally of the scope.

16. The method of claim 14, further comprising expanding two or more balloons from a distal portion of the access device.

17. The method of claim 16, further comprising stabilizing the access device within the body wherein using at least one balloon.

18. The method of claim 16, wherein the balloon is transparent and encases the scope when deployed.

19. The method of claim 12, wherein the material is a compliant membrane and the act of expanding comprises expelling a gel from the access device against the membrane.

20. The method of claim 19, wherein the membrane and the gel are both clear.

21. The method of claim 12, further comprising rupturing the expandable membrane.

22. The method of claim 21, wherein the scope is used to rupture the expandable membrane.

23. The method of claim 21, wherein the instrument is used to rupture the expandable membrane.

24. The method of claim 19, further comprising expanding the membrane until it contacts the target site.

25. A system for accessing a target site within the body, the system comprising:
a cannula having at least one lumen;
a scope deliverable through the at least one lumen; and
an expandable material deployable from a distal portion of the cannula, wherein expanding the material creates a space within tissue adjacent the distal portion of the cannula wherein the space facilitates visualization by the scope.

26. The system of claim 25, wherein the expandable material is a balloon.

27. The system of claim 26, wherein the balloon, when expanded, has a donut configuration having a central opening through which the scope has an unobstructed view.

28. The system of claim 26, wherein the balloon is expandable laterally of the distal portion of the cannula.

29. The system of claim 25, wherein the material is rupturable to release an expansion medium.

30. The system of claim 29, wherein the expansion medium comprises a therapeutic agent.

31. The system of claim 29, wherein the expansion medium comprises a gel.

32. The system of claim 25, wherein the material comprises a membrane.

33. A system for accessing a target site within the body, the system comprising:
a cannula having at least one lumen having an open distal end;
a scope deliverable through the at least one lumen; and
an expandable material affixed to and covering the open distal end of the lumen.

34. The system of claim 33, wherein the expandable material is expanded by an expansion medium delivered through the cannula.

35. The system of claim 34, wherein the expansion medium comprises a therapeutic agent.

36. The system of claim 34, wherein the expansion medium is transparent.

37. The system of claim 34, wherein the expansion medium comprises a gel.

38. The system of claim 34, wherein the expansion medium comprises saline.

39. The system of claim 33, wherein the expandable material is a balloon.

40. The system of claim 33, wherein the expandable material is membrane.

41. The system of claim 33, wherein the expandable material is rupturable.

42. The system of claim 41, wherein the scope is configured to rupture the expandable material.
